(19) United States
(12) Patent Application Publication
(10) Pub. No.: US 2015/0244452 A1
(43) Pub. Date: Aug. 27, 2015

(54) Systems and Methods for Monitoring Individuals for Substance Abuse

(71) Applicant: 1A Smart Start, Inc., Grapevine, TX (US)

(72) Inventors: Wojciech Grohman, Little Elm, TX (US); James Ralph Ballard, Flower Mound, TX (US)

(21) Appl. No.: 14/624,353

(22) Filed: Feb. 17, 2015

Related U.S. Application Data
(60) Provisional application No. 61/942,841, filed on Feb. 21, 2014.

(51) Int. Cl. H04B 7/26; G08B 25/00; G01N 33/497

(52) U.S. Cl. CPC H04B 7/26; G01N 33/497; G01N 33/4972; G08B 25/006

(57) ABSTRACT

A system for monitoring an individual for substance use includes at least one of a transdermal sensor and a body communications subsystem. When provided, the transdermal sensor is used to periodically test for consumption of a selected substance by the individual, the results of which are selectively logged for compliance review by monitoring authorities. When provided, the body communications subsystem uses the body of the individual as a communications path for signals transmitted by a transmitting device storing identification information for the individual to a receiving device for extracting the identification information and identifying the individual.

### CROSS-REFERENCE TO RELATED APPLICATION

The present application claims the benefit of U.S. Provisional Application Ser. No. 61/942,841, filed Feb. 21, 2014, and incorporated herein by reference for all purposes.

### FIELD OF INVENTION

The present invention relates in general to sobriety testing techniques, and in particular to systems and methods for monitoring individuals for substance use.

### BACKGROUND OF INVENTION

Sobriety testing, which includes testing for both alcohol and illegal drugs, has taken a prominent role in ensuring a safe and efficient society. For example, ignition interlocks on vehicles have proven their worth in preventing intoxicated drivers from entering the roadways and causing serious, including fatal, accidents. Sobriety testing has also allowed authorities, such as courts and law enforcement agencies, to monitor compliance with the court-ordered restrictions imposed on persons having committed alcohol or drug related offenses. Among other things, with the availability of reliable sobriety testing systems, such offenders can continue travel to work, school, or rehabilitation and thus contribute to society, rather than be a burden.

Sobriety interlock systems can often be circumvented, for example by having a sober individual take the sobriety test or by using a balloon or other source of intoxicant-free air. Hence, a number of techniques have been developed for reducing the probability of test circumvention, including the use of cameras to positively identify the person taking the sobriety test. Cameras also allow for the identification of an intoxicated person failing a sobriety test, such that the conduct of the identified individual can be reported to the monitoring authorities. However, cameras are not infallible and having a backup or complementary identification system could significantly improve the reliability of a sobriety interlock system.

Also known in the art are transdermal alcohol detection systems, which monitor for intoxication by measuring the amount of alcohol within the perspiration of the monitored driver. These systems have so far had limited value in monitoring vehicle operators, given the lag between the time the operator consumes alcohol and the time that alcohol is present at the surface of the skin. In other words, the vehicle operator could have started the vehicle and begun to drive well before the alcohol is detected. Nonetheless, transdermal alcohol detection systems could still have a valuable role to play in the reduction of intoxicated driving.

### SUMMARY OF INVENTION

According to one representative embodiment of the principles of the present invention, a monitoring system is disclosed for monitoring for consumption of a selected substance by a vehicle operator. A monitoring device, which includes a transdermal sensor for monitoring for the selected substance, attaches to a vehicle operator. A receiver, preferably mounted on a vehicle, receives data representing test results from the transdermal sensor. Processing circuitry, associated with the transdermal sensor processes the test results from the transdermal sensor and selectively logs a positive detection of consumption of the selected substance by the vehicle operator.

This embodiment allows the authorities to detect instances when the wearer of a transdermal sensing device operates a vehicle after having consumed an intoxicating or controlled substance such as alcohol, cannabis, prescription drugs, or illicit drugs. Consumption of the given substance is then logged and available for subsequent compliance review of the individual. Furthermore, a positive detection of the given substance by the transdermal sensor can be used to trigger the immediate transmission of a warning message to the authorities such that appropriate action can be taken. When used in conjunction with breath testing-based sobriety interlock system, a detection of positive alcohol consumption can be used to force a breath retest and/or disablement of the vehicle.

According to another representative embodiment of the principles of the present invention, a system is disclosed for identifying a vehicle operator. A first body communications device transmits signals carrying information identifying a vehicle operator using the body of the vehicle operator as a communications path. A second body communications device receives the signals carrying the information identifying the vehicle operator from the body of the vehicle operator. Processing circuitry then processes the received signal to positively identify the vehicle operator.

This embodiment provides a compact and secure way of identifying an individual requiring testing for use of alcohol or a controlled substance. It can be employed with both home substance testing apparatus and in-vehicle substance testing apparatus, including those found in vehicle sobriety interlock systems. Because these systems use the human body as a transmission medium, the ability to intercept communications between the components of the system is substantially reduced. Message encryption further enhances security.

### BRIEF DESCRIPTION OF DRAWINGS

For a more complete understanding of the present invention, and the advantages thereof, reference is now made to the following descriptions taken in conjunction with the accompanying drawings, in which:

FIG. 1A is a diagram of a portion of an interior of a vehicle including a sobriety interlock system and vehicle operator identification system suitable for demonstrating one possible application of the principles of the present invention;

FIG. 1B is a high level functional block diagram of the exemplary sobriety interlock system and vehicle operator identification system utilized in the application shown in FIG. 1A;

FIG. 2 is a more detailed functional block diagram showing the primary subsystems of the handheld unit shown in FIG. 1B;

FIG. 3 is a flow chart illustrating a preferred procedure for using a transdermal detection device, alone or in combination with the interlock system shown in FIGS. 1A and 1B, for identifying instances of driving under the influence of alcohol or another controlled substance according to one embodiment of the principles of the present invention; and

FIG. 4 is a flow chart illustrating a preferred procedure for using a body communications system, alone or in combination with the interlock system shown in FIGS. 1A and 1B, for identifying a vehicle operator according to another embodiment of the principles of the present invention.

### DETAILED DESCRIPTION OF THE INVENTION

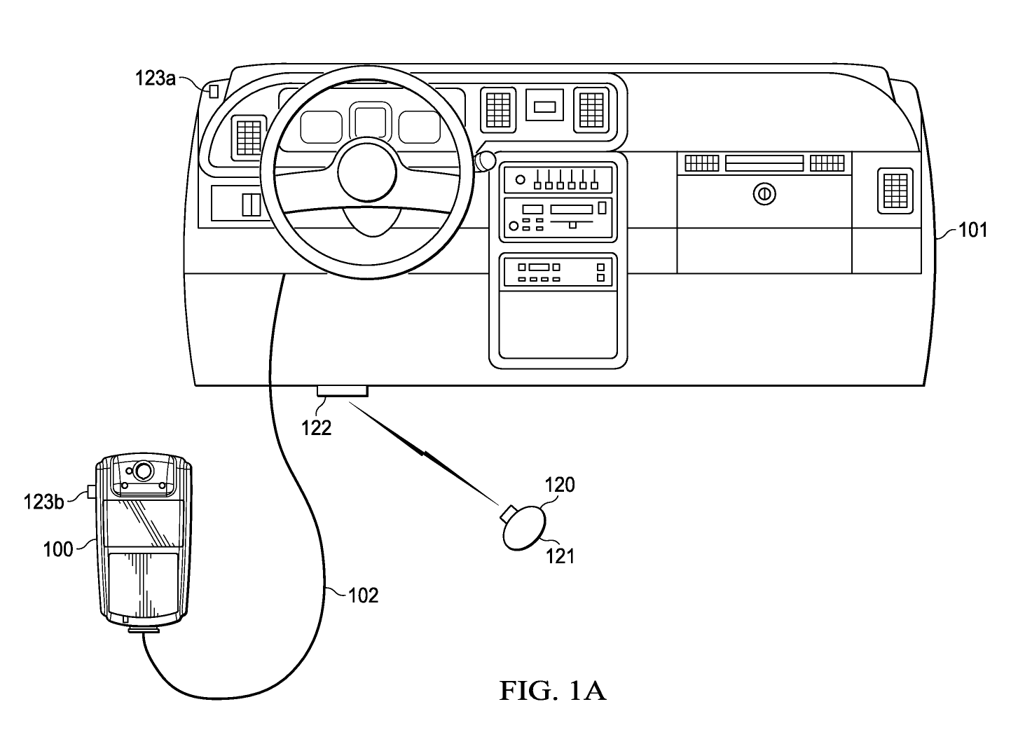

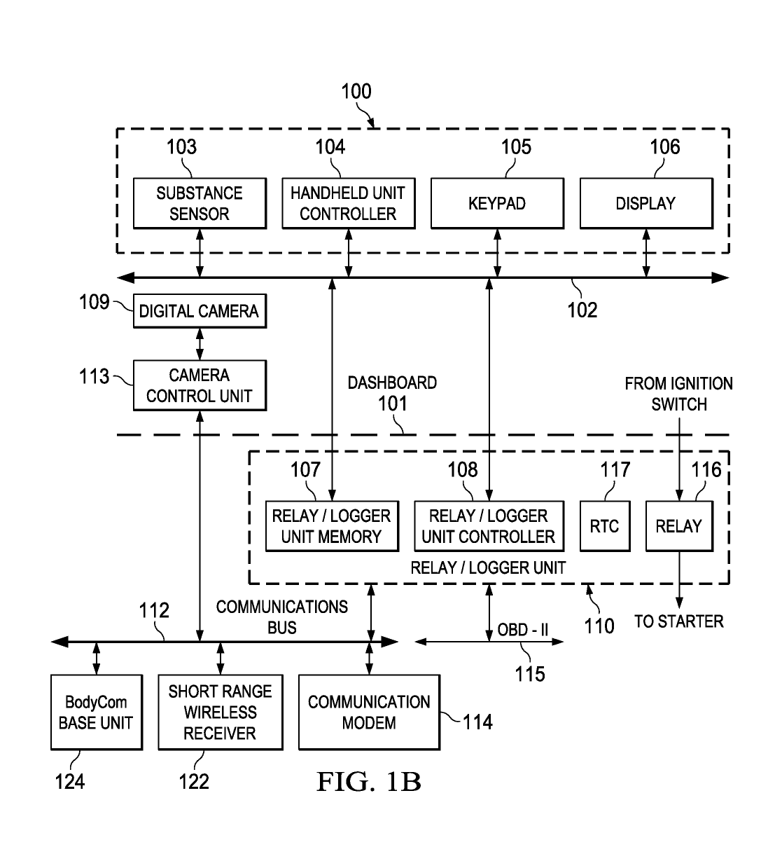

The principles of the present invention and their advantages are best understood by referring to the illustrated embodiment depicted in FIGS. 1-4 of the drawings, in which like numbers designate like parts. For discussion purposes, these principles will be described in conjunction with an alcohol breath testing system operating within a vehicle ignition interlock system. It should be recognized, however, that the systems and methods described below are equally applicable to other types of sobriety testing systems, including stand-alone sobriety testing systems and those designed to test for other types of intoxicants and controlled substances (e.g., marijuana).

FIG. 1A is a diagram showing a portion of the interior of a motor vehicle in the area of the dashboard. A handheld breath alcohol testing unit 100 is connected to electronic circuitry behind vehicle dashboard 101 (see FIG. 1B) through a cable 102. Generally, a person attempting to start the vehicle must provide a breath sample to handheld unit 100, which tests for deep-lung breath alcohol content, deep-lung alcohol content being directly proportional to blood alcohol concentration and thus intoxication level. If the person being tested passes the breath alcohol test, the interlock system allows the vehicle to start. On a test failure, the interlock system disables the vehicle ignition system and the vehicle is rendered inoperable.

FIG. 1B is a high level functional block diagram of the overall interlock system. Handheld unit 100, which is discussed in detail below, includes a substance sensor 103, which in the illustrated embodiment is based on a fuel cell alcohol sensor and includes its assembly, a handheld unit controller 104, a keypad 105 for data entry, and a display 106.

Handheld unit 100 electrically communicates through cable 102 with electronics behind dashboard 101. The electronics behind dashboard 101 include relay/logger unit 110, which comprises relay/logger unit memory 107 and relay/logger unit controller 108. Relay/logger unit memory 107, which is preferably solid state memory, such as Flash memory, stores the results of tests performed by handheld unit 100 for periodic retrieval and review by authorities monitoring the driver for compliance with any conditions or restrictions imposed on the driver. In addition, relay/logger unit memory stores the firmware controlling the operation of relay/logger unit controller 108.

Relay/logger unit controller 108, operating in conjunction with handheld unit 100, controls the operation of the vehicle in response to the outcome of a given test. As known in the art, the ignition system of a vehicle can be controlled in any one of a number of ways, including enabling or disabling relays providing power to the starter motor or sending enable or disable commands to one or more on-board computers. In the illustrated embodiment, relay/logger unit controller 108 controls a relay 116, which in turn controls the flow of electrical current between the vehicle ignition switch and the starter motor. Relay/logger unit controller 108 may also be used to generate visible or audible warnings in the event of a failed test, for example, causing the horn to sound or the headlights to flash.

A digital camera 109 or similar imaging device is also preferably provided to allow for positive identification of the person taking the breath test through handheld unit 100. The images taken by digital camera 109 are preferably stored in relay/logger unit memory 107 and/or memory associated with Camera Control Unit 113 for retrieval and review by the monitoring authorities. Advantageously, digital camera 109 reduces the possibility of a restricted or intoxicated driver of circumventing the interlock system by having a substitute person providing the breath sample to handheld unit 100. In the illustrated embodiment, digital camera 109 operates in conjunction with a camera control unit 113, which communicates with relay/logger unit controller 108 via a communications bus (such as an RS-485 standard bus, Ethernet or CAN standard bus) 112.

Also operating off of communications bus 112 is a cellular telecommunications modem 114, which allows relay/logger unit controller 108 to wirelessly send alerts to the authorities in the event of a failed test (i.e., the detection of a controlled substance) or transmit logged information within relay/logger unit memory 107 to the monitoring authorities, whether or not an intoxicated driver has been detected.

In one particular embodiment, handheld unit 100, relay/logger unit memory 107, relay/logger unit controller 108 communicate, either in whole or in part, with the OBD-II diagnostic system 115 standard on most motor vehicles. The OBD-II system provides another efficient mechanism by which monitoring authorities can access the data stored within relay/logger unit memory 107 through a standard OBD-II port and associated test equipment. In addition, the OBD-II also allows for vehicle operating data to be recorded and stored within relay/logger unit memory 107 for correlation with the results of sobriety testing performed through handheld unit 100.

The OBD-II diagnostic system also provides a communications path for transmission of command and control signals from relay/logger unit controller 108 to various electronics and electrical systems within the vehicle. These command and control signals can be used by interlock system controller 104 to disable the vehicle in response to a failed intoxication test.

In the illustrated embodiment, relay/logger unit controller 108 includes a microprocessor or microcontroller, such as a Renesas R5F3650NDFB or similar device. A real time clock 117, such as a Seiko S-35390A, operating in conjunction with relay/logger unit controller 108, tracks the date and time.

According to the principles of the present invention, a monitored person wears a device 121 which could be a bracelet mounted around the ankle or wrist or a necklace mounted around the neck, which supports a tamper-proof monitoring device 120, as shown in FIG. 1A. As discussed in detail below, in one embodiment, monitoring device 120 includes a transdermal alcohol sensor and wireless transmitter, such as those found in a SCRAM monitoring device available from Alcohol Monitoring Systems, Inc. or similar commercially available substance monitoring system. In another embodiment, monitoring device 120 includes a BodyCom mobile unit, which is commercially available as part of the BodyCom system of Microchip Technology, Inc.

For those embodiments of monitoring device including a transdermal alcohol sensor, a short range wireless receiver 122 is provided in the driver's side foot well underneath dashboard 101 for wirelessly communicating with monitoring device 120. As shown in FIG. 1B for the illustrated embodiment, wireless receiver 122 preferably communicates with relay/logger unit 110 through communications bus 112. For those embodiments using the BodyCom system discussed in detail below, at least one capacitive touch pad 123 is provided, for example capacitive touchpad 123a disposed on or around the dashboard 101 or capacitive touch pad 123b disposed on handheld testing unit 100. Capacitive touch pad 123 communicates with a BodyCom system base unit 124 coupled to communications bus 112 and relay/logger unit 110, as shown in FIG. 1B.

Preferably, the capacitive touch pad 123 is mounted in a place requiring touching before or while operating the vehicle, such as the push-start button or the steering wheel. Similarly, the touch pad 123b is preferably mounted in a place requiring direct touch with the human body, such as where the hand grip is located or around the mouthpiece in such a location that test subject's lips touch it while giving a breath sample. These specific locations enhance the system's security and prevent obvious circumventions of the test system.

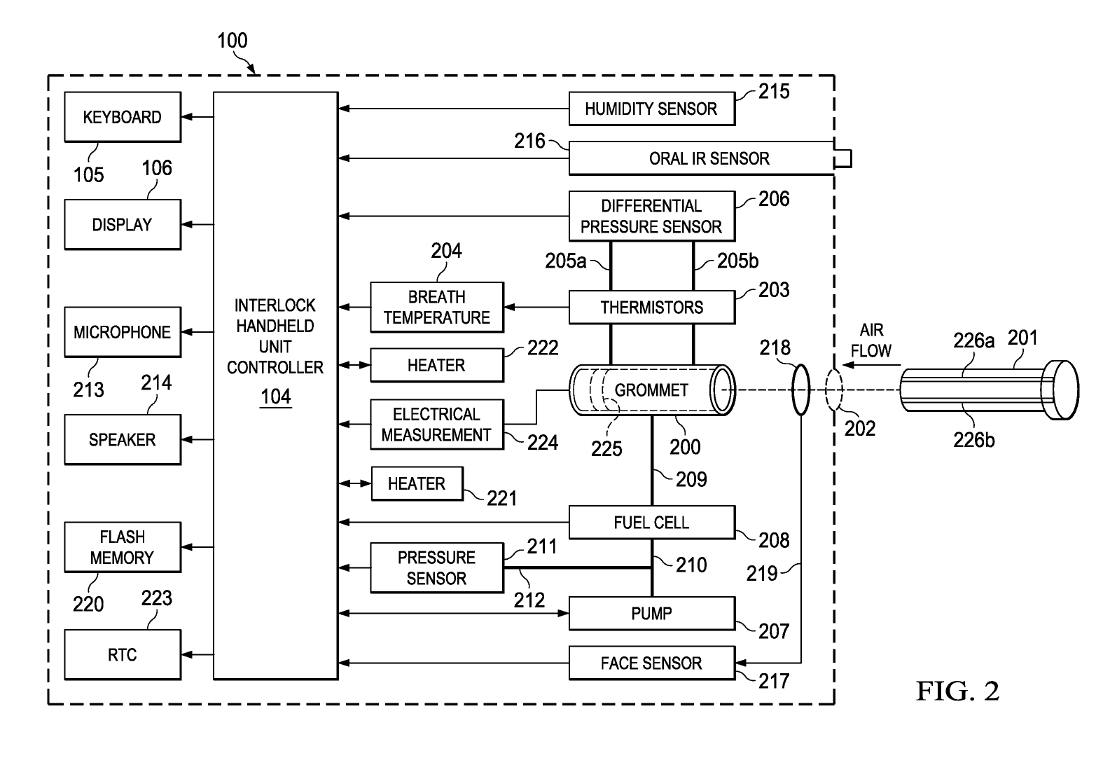

FIG. 2 is a more detailed functional block diagram of the primary subsystems within handheld unit 100 in a preferred embodiment of the principles of the present invention. In this embodiment, interlock system controller 104 is a Renesas R5F3650NDFB processor operating in conjunction with firmware stored in Flash memory 220. For clarity, interface devices, such as the analog to digital converters (ADCs) interfacing the various blocks with controller 104, and auxiliary subsystems, are not shown in FIG. 2.

A cylindrical grommet 200 receives a disposable mouthpiece 201 through an aperture 202 through the front panel of the case of handheld unit 100. Air introduced by a user (i.e., the human test subject) through mouthpiece 201 generally passes through cylindrical grommet 200 and passes out an aperture through the unit rear panel.

As air flow passes through grommet 200, a set of at least one thermistor 203 and associated breath temperature measurement circuitry 204 measure breath temperature. Breath temperature is one parameter useful for detecting attempts to circumvent an alcohol breath test.

A pair of tubes 205a-205b tap the airflow through grommet 200 to a differential pressure sensor 206, which measures breath pressure and breath air flow rate. As known in the art, in order for an alcohol breath test to be valid, the user must provide sufficient air pressure for a sufficiently long period of time to ensure that a deep-lung air is received by the alcohol sensor. If neither of these two conditions is met, interlock system controller 104 aborts the test and the breath test functional routine is reset. One device suitable for use as differential pressure sensor 206 in the embodiment of FIG. 2 is a Sensormatic 35AL-L50D-3210 differential pressure transducer.

Once interlock system controller 104 determines that deep-lung air is being received, a pump 207 is activated to draw a sample of the air flowing through grommet 200 into a fuel cell 208. In the illustrated embodiment, the air sample is drawn through tubes 209 and 210. A pressure sensor 211 monitors the air pressure being provided by pump 207 through a tube 212. One suitable fuel cell 208 is a Dart Sensors LTD 2-MS3 fuel cell operating in conjunction with a pump 207 available from PAS International, although other commercially available fuel cells and pumps may be used in alternative embodiments. A suitable device for pressure sensor 211 is a Sensormatic 33AL-L50D-3210 pressure transducer.

Fuel cell 207 implements a well-known electrochemical process to determine the breath alcohol content of the deep-lung air sample. From the air sample, interlock system controller 104 calculates the corresponding blood alcohol concentration and determines whether the user has passed or failed the test, depending on the legal limits imposed by the given jurisdiction. In response to the test result, interlock system controller 104 sends commands to vehicle electronics/electrical system 108 to enable or disable the vehicle ignition system. The results of the test are also recorded within relay/logger unit memory 107 for access by the monitoring authorities.

The user interacts with system controller 104 through keypad 105 and display 106, which allow the user to receive prompts and initiate a test in anticipation of starting the vehicle. In addition, interlock system controller 104 may periodically require retest of the user to ensure driver sobriety after initial start of the vehicle. In alternate embodiments, a microphone 213 and speaker 214 allow for control of handheld unit 100 by voice command.

In the illustrated embodiment of handheld unit 100, multiple sensors are provided for preventing circumvention of the breath test. In addition to breath temperature circuitry 204, handheld unit 100 also includes a humidity sensor 215, an oral infrared (IR) sensor 216, and a face proximity sensor 217. In the embodiment shown in FIG. 2, face proximity sensor 217 operates in conjunction with an electrode 218 disposed on the inner surface of the front panel of the case of handheld unit 100 and at least partially surrounding aperture 202. A clip 219 provides an electrical connection between the printed circuit board on which face proximity sensor circuit 217 resides and electrode 218.

Temperature can have a significant effect on the operation of handheld unit 100 at cold or very cold temperatures. Among other things, the speed of the electrochemical reaction within fuel cell 208 typically decreases with decreasing temperature. In addition, fuel cell 208 also is subject to a temperature coefficient, wherein the strength of the generated detection signal decreases with decreasing temperature. In addition, when grommet 200 is cold, condensation from the test subject's breath can adversely impact the test measurement.

In order to ensure proper breath content measurements are taken, grommet 200 is heated by a heater 222, which is, for example, one or more metallic sheets disposed around the grommet outer periphery. Similarly, a heater 221 maintains the temperature of fuel cell 208. Heater 221 may be, for example, a metallic sheet disposed against one or more of the outer surfaces of fuel cell 208 or a metal block on which fuel cell 208 sits. In embodiments of handheld unit 100 using a Renesas R5F3650NDFB microcomputer, heaters 221 and 222 are driven with pulse width modulated (PWM) signals that can be made available at certain controller input/output pins by firmware programming. In addition, the temperature of fuel cell heater 221 and grommet heater 222 are monitored and corresponding signals returned to handheld unit controller 104.

Continuous alcohol monitoring systems, which typically measure the amount of alcohol exuded in human perspiration, are known in the art. Generally, the person being monitored wears a monitoring device, typically in the form of a non-removable bracelet, twenty four hours a day, seven days a week. The monitoring device periodically samples the wearer's perspiration and tests its alcohol content. The results are logged and can be wirelessly transmitted to the monitoring authorities using an integral wireless communications system or downloaded to a computer for subsequent transfer and review. The SCRAM System, marketed by Alcohol Monitoring Systems, Inc., is one example of commercially available continuous alcohol monitoring system.

One significant drawback of perspiration monitoring is the transdermal absorption time. Depending on the individual wearer, the amount of alcohol consumed, and the environmental conditions, the time between consumption of alcohol and the resulting positive detection of alcohol by the wearer and detection of alcohol by the monitor may be 30 minutes or more. In the interim, the wearer may have started and begun to operate a motor vehicle. In the case where no sobriety interlock system is provided, the wearer may have simply started the vehicle while intoxicated, either knowingly or unknowingly, and begun to drive. In the case of a vehicle including a sobriety interlock system, the wearer may have successfully circumvented the sobriety test by having another person provide the breath sample or by using a balloon of alcohol-free air.

As indicated above with regards to FIGS. 1A and 1B, in one embodiment of the principles of the present invention, monitoring device 120 includes a transdermal alcohol sensor and wireless transmitter and is tethered to a monitored person with a bracelet 121 or similar tamper-resistant device. Monitoring device 120 periodically monitors the perspiration of the monitored person and transmits those results through short range wireless receiver 122 and relay/logger unit 110 for logging.

Due to the time lag between the consumption of alcohol and the ability to detect that alcohol at the skin, a person with transdermal monitoring may still be able start and operate a vehicle. Hence, in a vehicle that does not have a conventional breath-based interlock system, the primary purpose of the transdermal detection embodiment of monitoring device 120 is to detect and positively identify an intoxicated driver. This information can then be subsequently retrieved from relay logger unit 110 during compliance review of the monitored person's conduct or be transmitted immediately through wireless communications modem 114 such that the authorities can take appropriate action. In addition, a picture can be taken of the driver, for example using digital camera 109 in the system described above.

If an interlock device, such as the breath testing-based system described above, is also provided, then the transdermal detection embodiment of monitoring device 120 is used to confirm the results of the breath testing, prevent circumvention of the breath test by an intoxicated driver, or identify a driver who has consumed alcohol after successfully passing the breath test and starting the vehicle. For example, if the driver successfully passes the breath test and begins to operate the vehicle, but a subsequent transdermal test reveals the presence of alcohol, the driver can be immediately forced to take the retest. In the driver fails the breath retest, the interlock system disables the vehicle's ignition system and optionally, notifies the authorities through the wireless communication modem 114. In the event the driver passes the breath test, the driver can be allowed to continue to operate the vehicle, but the results of the transdermal detection are logged by relay/logger unit 110 for compliance monitoring purposes. Alternatively, if the results of the breath test and the transdermal detection are in conflict, the vehicle may be disabled by the interlock system until both the transdermal detector and the breath tester results correlate or periodic breath retesting may be required, depending on the testing protocol in place.

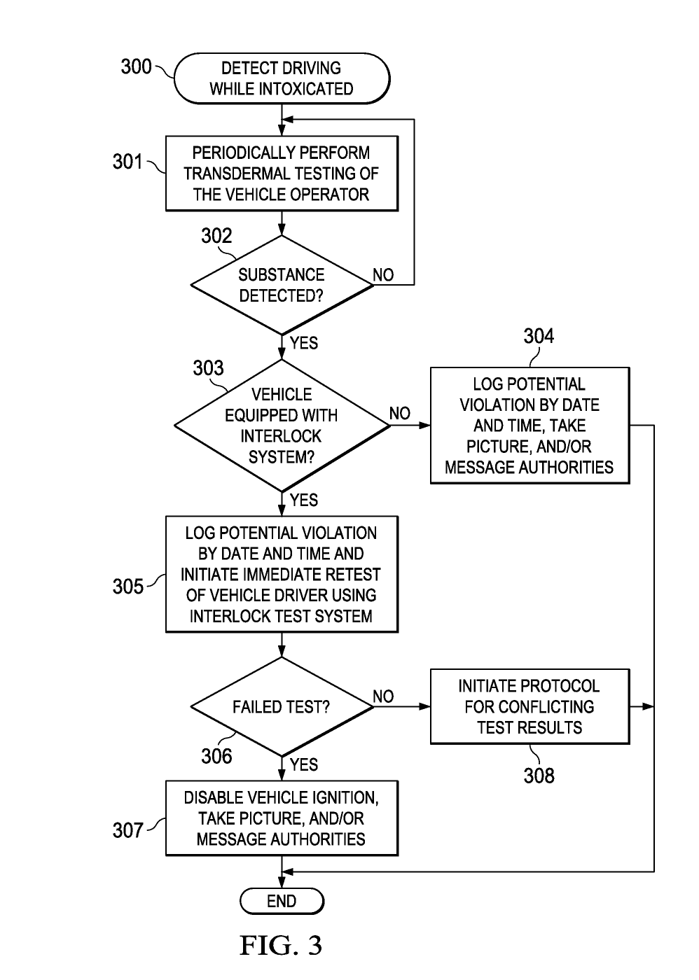

FIG. 3 is a flow chart of a procedure 300 for detecting driving while intoxicated using a transdermal substance detector. At Block 301, periodic testing of the vehicle operator is performed for the presence of alcohol or other controlled substance. If alcohol or another controlled substance is not found at Decision Block 302, then no action is taken and the monitoring process continues so long as the vehicle operator remains in the vehicle (e.g., is within the operating range of wireless receiver 122.)

On the other hand, if alcohol or a controlled substance is detected, then one of at least two courses of action are taken at Decision Block 303. If the vehicle is not equipped with another substance test system, such as the breath-based system discussed above, then at Block 304, the potential violation is logged by date and time (e.g., by relay/logger unit 110), a picture is taken of the driver (e.g., by digital camera 109), and/or the authorities are messaged such that appropriate action can be taken (e.g., using modem 114).

If the vehicle is equipped with an interlock system, then the results from the transdermal detector may still be logged for later analysis, but a retest of the driver is immediately initiated at Block 305 (e.g., using hand-held unit 100). If the driver fails the test at Block 306, then the interlock system takes preventive action to disable the vehicle, a picture is taken of the driver, and/or the authorities are notified (Block 307). The preventive action taken by the interlock system depends on the vehicle's state during the test. In particular, if the vehicle's engine is already running, the interlock system may prevent a subsequent engine turn on, or it may communicate with the vehicle's powertrain controls to request a gradual (safe) slowdown of the vehicle until it reaches a minimum speed or stops completely. If the vehicle's engine is not running during the test, the interlock system prevents the engine from starting, normally by opening the relay 116 to prevent the engine's starter from working. In general, the term disabling the vehicle will refer to any action taken to prevent the vehicle's use, either immediately, or as soon as otherwise possible.

If the driver passes the test at Block 306, then at Block 308 a protocol for conflicting test results is applied. The driver may be allowed to continue to operate the vehicle, for example because the amount of alcohol detected is below the legal limit for operating a vehicle, although the potential violation of conditions placed on the driver may still be logged. Alternatively, the vehicle may be disabled until the results of transdermal testing and breath testing agree or the driver may be allowed to continue operating the vehicle with increased periodic retesting by the breath tester.

According to a second embodiment of the principles of the present invention, tethered monitoring device 120, 121, which could be a SCRAM bracelet or its equivalent, includes an alcohol monitoring device and integral long range wireless communications device. In this embodiment, a vehicle interlock system, for example, handheld unit 100, relay/logger unit 110, and communications modem 114 in the system discussed, above is also used. Both tethered monitoring device 120, 121 and the vehicle interlock system (e.g., relay/logger unit 110, communications modem 114) communicate with the server of the monitoring authorities to transfer two sets of data representing independently-taken substance testing results. The results from the tethered monitoring device 120, 121 and the vehicle interlock system are later compared for consistency.

In addition, tethered monitoring device 120, 121 includes a short range wireless transmitter and the vehicle interlock system includes a short range wireless receiver (e.g., short range receiver 122), which work together to determine if the person driving the vehicle is wearing a tethered monitoring device. Preferably, a driver wearing tethered device 120, 121 is identified by identification data stored in the tethered device itself. This identification data is periodically transmitted to the vehicle's interlock system and logged to create a record of any driver with a tethered device or its equivalent who drove the vehicle at any given time. While this embodiment does not actively prevent an intoxicated person from driving the vehicle, it allows the monitoring authority to determine if the person wearing the tethered device engaged in activities that are dangerous after the fact and allows them to take immediate action to prevent the person from such activities in the future. One such preventive measure is to restrict the use of the vehicle by the person wearing the tethered device to only certain times of the day and week or to a geographical boundary defined by GPS coordinates or calculated by cell tower triangulation and retrieved from communications modem 114.

As indicated above, another embodiment of monitoring device 120 includes a BodyCom mobile unit, which communicates with a BodyCom base unit 124, as shown in FIGS. 1A and 1B. The body communication technology or “BodyCom” is a relatively new technology developed and made commercially available by Microchip Technology, Inc. The basics of BodyCom are described in the Microchip Technology publication AN 1391 Introduction to BodyCom Technology, incorporated herein by reference (“the Microchip Paper”).

The BodyCom technology uses the human body as a transmission medium for the short-range wireless exchange of radio frequency (RF) signals. Generally, it was found that relatively simple electrical circuit could successfully generate and transmit RF signals through the human body in the frequency range of 60 kHz to 30 MHz. One significant advantage of this system is security, given that no RF signals are transmitted through the air where they can potentially be intercepted.

In the human body, amplitude attenuation increases with lower frequencies such that the required transmission power increases with decreasing frequency. Therefore, the BodyCom system typically uses a larger base unit, which can be either externally powered or powered with a larger battery, transmitting at a lower frequency (e.g., 125 kHz) and a smaller battery-powered mobile unit, transmitting at a higher frequency (e.g., between 6 and 13 MHz). Packetized data are exchanged between the base and mobile units using an amplitude shift key (ASK) modulation format, such as on/off keying (OOK).

The exemplary base unit described in the Microchip Paper includes a microprocessor operating in conjunction with transmit and receive paths similar to those found in conventional RF wireless communications systems. The transmit path includes a data signal modulator (DSM), an RF driver, and a touch pad/capacitive coupling pad communicating with the RF driver. The receive path includes a pre-amplifier, also coupled to the touch pad, an RF mixer operating from a local oscillator, and a data demodulator. The mobile unit is similar and includes a microprocessor operating in conjunction with a transmit path, including a DSM and a data transmitter communicating with a capacitive coupling pad, and a receive path, including an RF receiver and demodulator unit communicating with the capacitive coupling pad.

Generally, each microprocessor generates packetized data using the Universal Serial Asynchronous Receiver/Transmitter (UART) standard format. The packetized data may be encrypted, for example using the Advanced Encryption Standard (AES) and then modulated by the associated DSM. The modulated data are then capacitively coupled to the human body through the associated capacitive coupling pad. At the receiving end, the data are down-converted in frequency, as necessary, demodulated, and processed by the given microprocessor.

A typical transaction using the BodyCom system starts when the base unit detects either a touch on the base unit touch pad or a human body in close proximity thereto (i.e., within 1 cm). The base unit microprocessor then generates a packet including a wake-up pulse and set-up information for the mobile unit, as well as the actual data to be transmitted. After the data are modulated, they are transmitted by an RF driver and the base station capacitive coupling pad through the user's body. The packet is then capacitively coupled by the mobile unit capacitive coupling pad to the mobile unit receiving circuitry and microprocessor.

In response, the mobile unit wakes-up and decodes the data. The mobile unit microprocessor generates an answer data packet, which is modulated and capacitively coupled through the mobile unit capacitive coupling pad and transmitted through the user's body to the base unit. In systems where the mobile unit is transmitting at 8 MHz, the packet received by the base unit is down-converted in frequency with the mixer, demodulated, and decoded.

Multiple cycles may be used to transfer information between the base and mobile units, as necessary.

According to the principles of the present invention, the mobile BodyCom unit within monitoring device 120 provides a positive identification of the operator of the vehicle operator, in lieu of, or in addition to, other identification devices such as digital camera 109. Advantageously, because signals between monitoring device 120 and base unit 124 travel through the body of the monitored person, the chance of interception by third parties is significantly reduced. Encryption of the information being exchange may can also be used to further increase the security of the system.

Moreover, the BodyCom technology allows monitoring device 120 to be implemented in a small package fastened to an accordingly small bracelet or similar support structure 121. For example, monitoring device 120 and bracelet 121 could together be worn around the user's wrist, ankle, as a necklace, or as part of a wristwatch. In addition, monitoring device 120 could be packaged in the form of a small tag, which could be fastened to, or incorporated in, the user's car keys or even fastened to the user via a biocompatible adhesive.

Monitoring device 120 can not only be used to identify an individual requiring monitoring, but can also be used to differentiate between individuals requiring testing before being allowed to drive and individuals who do not require testing before being allowed to drive. Current vehicle interlock systems typically do not differentiate between vehicle operators requiring testing and those who do not. As a result, anyone attempting to start the vehicle must normally be subjected to the substance testing procedure, whether or not warranted.

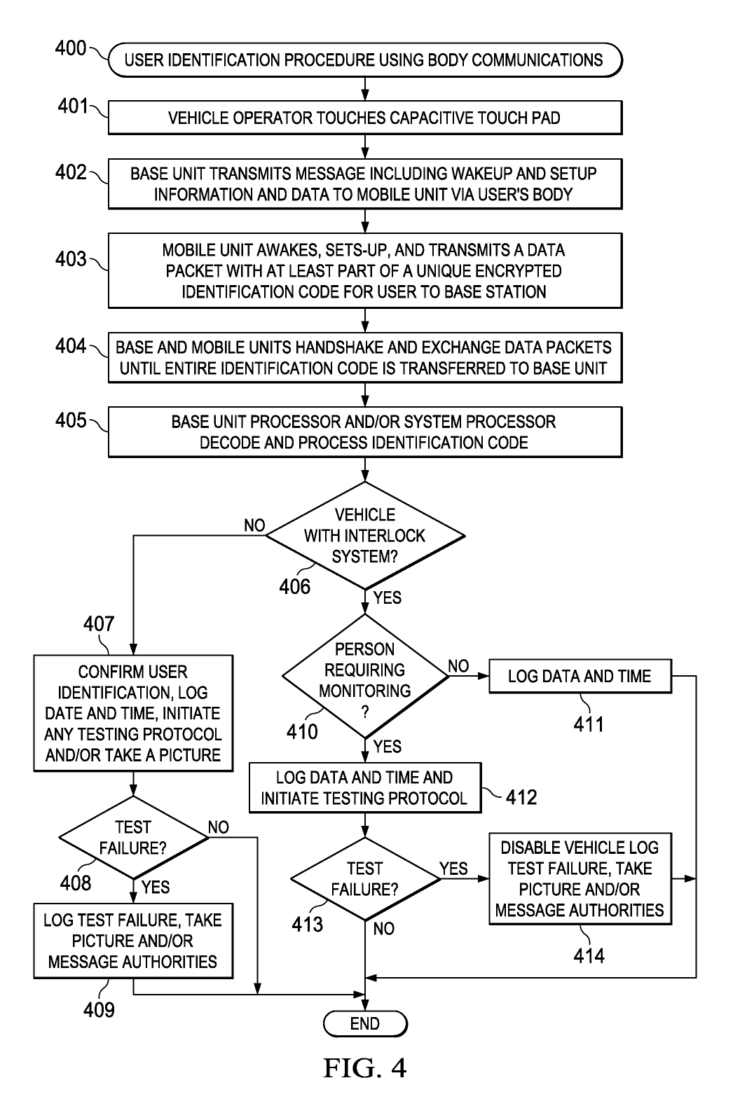

A preferred Procedure 400 shown in FIG. 4 illustrates one possible way of effectively using the BodyCom-based embodiment to identify a person subject to monitoring. While Procedure 400 uses the identification of a vehicle operator as an example, the principles of the present invention are equally applicable to other types of systems requiring identification of an individual, such as home alcohol and drug testing systems.

At Block 401, the vehicle operator touches a base unit capacitive keypad (e.g., capacitive touch pad 123a on dashboard 101 or capacitive touch pad 123b on hand-held unit 100 in the system shown in FIGS. 1A and 1B). In response, base unit 124 transmits a data packet including wakeup and setup information and data to the mobile unit within monitoring device 120 via the user's body (Block 402).

The mobile unit within monitoring device 120 then returns a data packet including at least part of a unique ID of the user, which is preferably, but not necessarily, encrypted (Block 403). At Block 404, base unit 124 and the mobile unit within monitoring device 120 handshake, as necessary, to transfer the entire user ID to base unit 124.

The processor of base unit 124 and relay/logger unit controller 108 decode and process the identification code at Block 405. As previously indicated, Procedure 400 is applicable to both a system with a sobriety interlock system, such that described above, or with sobriety testing system that does not include a vehicle interlock, such as a home alcohol or drug testing system or a vehicle-based system that requires the identification of the operator, but does not perform sobriety testing. In FIG. 4, this option is represented by Decision Block 406.

In the case of a system that does not include an interlock system, then at Block 407, the identification of the user is confirmed, the date and time are logged, picture may be taken and any required testing protocol is initiated (e.g., a home drug or alcohol test). In the event a test is required, and the user passes that test (Block 408), then Procedure 400 ends. On the other hand, if the user fails the substance test, the failure is logged, a digital picture is taken, and/or a message is sent to the authorities, as appropriate.

For systems including an vehicle sobriety interlock system, then the user identification is used at Block 410 to determine if the vehicle operator is an individual requiring monitoring or testing. If not, the time and date may be logged and Procedure 400 is complete. Otherwise, at Block 412, the date and time are logged and the test protocol is initiated, for example using hand-held unit 100 described above. If the monitored individual takes and passes the test, at Block 413, Procedure 400 is complete. However, if the monitored person takes and fails the test, the failure is logged, the vehicle is disabled, a picture is taken of the person attempting to operate the vehicle, and the authorities are messaged, as appropriate.

Monitoring device 120 may include both a transdermal alcohol sensor, as well as a BodyCom identification system. In this case, the BodyCom identification system is used to confirm the identification of the individual attempting to start and operate the vehicle.

A monitored user may attempt to avoid identification or start the vehicle while intoxicated by having a second person take the breath alcohol test. To minimize the risk of circumvention, at least some embodiments of handheld unit 100 are provided with an electrical measurement circuit 224, which detects changes in the electrical characteristics of a received signal, such as current, voltage, and/or power. In some embodiments, measurement circuit 224 may also be able to measure the total capacitance between the transmitter and a receiver and any signal indicative of the received signal strength, a measure commonly referred to Received Signal Strength Indicator (RSSI).

In the preferred embodiment, electrical measurement circuit 224 receives its input from an electrical contact or electrode 225 within the bore of grommet 200 and provides an output signal to handheld unit controller 104 for processing. Electrical contact 225 may be a metallic strip or ring extending around the inner surface the grommet bore, one or more metallic strips extending along the longitudinal axis of the grommet bore, or a single metal contact, to name only a few options.

Electrical contact 225 within the grommet bore receives and establishes an electrical connection with one or more electrical contacts 226 on mouth piece 201. In the illustrated embodiment, two metallic strips 226a and 226b are shown on mouthpiece 201 for reference, although in alternate embodiments, the metallic mouthpiece contact may be a single strip or similar structure that provides both a contact with grommet bore contact 225 and to the mouth of the person taking the breath alcohol test. Electrical contacts 225 and 226 form the capacitive pad used to couple the communications to and from the human body.

During operation, the monitored person wearing the mobile body comm unit within monitoring device 120 touches the capacitive pad (e.g., pad 123a on the vehicle dashboard or pad 123b on handheld unit 100) during the test by handheld unit 100. If the monitored person is the one taking the test, then the characteristics of the RF electrical signals received by electrical measurement circuit 224 from body comm base unit 124 and/or monitoring device 120 will have a particular set of characteristics. (These particular characteristics are preferably set or trained for the monitored person.) For example, the current received by electrical measurement circuit 224 will have a range of values, depending on the physical characteristics of the person taking the test.

On the other hand, if a person other than monitored person takes the breath alcohol test using handheld unit 100, the electrical characteristics of the RF signal measured by electrical measurement circuit 224 will change. At least two different scenarios are possible: (1) the second person is not in contact with the monitored person during the breath alcohol test; and (2) the second person is in contact with the monitored person in an attempt to circumvent the system.

In the first case, the electrical characteristics measured by electrical measurement circuitry 224 will vary significantly from those expected when the monitored person is taking the test. For example, the amplitude of the signal received by monitoring circuitry 224 from body comm base unit 124 and/or monitoring device 120 will be very large but the capacitance will not change significantly from the expected value.

In the second case, with the second person in contact with the monitored person, the amplitude of the received signal will decrease over the scenario where the monitored person is taking the test and the capacitance may change, as well.

The identification of an individual for monitoring compliance with court-enforced driving restrictions is only one possible use of the body comm system described above. The body comm technology may be used in a vehicle without the interlock system described above, or even without an interlock system interlock system at all. Generally, a capacitive pad, similar to capacitive pad 123a on the dashboard and 123b on handheld unit 100, can be placed in one or more places around the interior of the vehicle for identifying any person carrying the corresponding mobile unit. For example, a capacitive pad could be placed on the steering wheel, a start button, shifter, seat or headrest, among other locations.

Although the invention has been described with reference to specific embodiments, these descriptions are not meant to be construed in a limiting sense. Various modifications of the disclosed embodiments, as well as alternative embodiments of the invention, will become apparent to persons skilled in the art upon reference to the description of the invention. It should be appreciated by those skilled in the art that the conception and the specific embodiment disclosed might be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore contemplated that the claims will cover any such modifications or embodiments that fall within the true scope of the invention.

What is claimed is:

1. A system for monitoring for consumption of a selected substance by a vehicle operator comprising:
a monitoring device for attaching to a vehicle operator, the monitoring device including a transdermal sensor for monitoring for consumption of the selected substance by the vehicle operator;
a receiver mounted on a vehicle for receiving data from the transdermal sensor; and
processing circuitry for processing data representing test results from the transdermal sensor and selectively logging a positive detection of consumption of the selected substance by the vehicle operator.

2. The system of claim 1, wherein the selected substance is selected from the group consisting of alcohol, cannabis, illicit drugs, and prescription drugs.

3. The monitoring system of claim 1, wherein the receiver comprises a wireless receiver for receiving wireless signals carrying the data representing the test results from the monitoring device.

4. The monitoring system of claim 1, wherein the receiver comprises a wireless receiver for receiving wireless signals carrying positive identification of the person wearing the said monitoring device.

5. The system monitoring system of claim 1, further comprising a wireless modem for selectively transmitting a message to a monitoring authority in response to a positive detection of consumption of the selected substance by the vehicle operator.

6. The monitoring system of claim 1, wherein:
the system further comprises a sobriety interlock system including:
a sobriety testing device for testing for alcohol in the breath of the individual being monitored; and
circuitry for disabling the operation of the vehicle in response to a positive detection of alcohol in the breath of the individual being monitored; and
the processing circuitry is further operable to initiate a test of the vehicle operator by the sobriety testing device of the interlock system when the transdermal sensor detects alcohol consumption by the individual.

7. The monitoring system of claim 1, further comprising:
a first body communications device disposed within the monitoring device for sending information identifying the vehicle operator using the body of the vehicle operator as a communications path; and
a second body communications device associated with the vehicle's interlock system for receiving and decoding the information identifying the vehicle operator transmitted from the first body communications device.

8. A method for monitoring for consumption of a selected substance by a vehicle operator comprising:
periodically testing for consumption of a selected substance by a vehicle operator using a transdermal sensor; and
selectively logging with processing circuitry a positive detection consumption of the selected substance by the vehicle operator.

9. The method of claim 8, wherein the selected substance is selected from the group consisting of alcohol, cannabis, illicit drugs, and prescription drugs.

10. The method of claim 8, further comprising wirelessly transmitting the test results from the transdermal sensor to the processing circuitry for logging.

11. The method of claim 8, further comprising transmitting a message with a wireless modem within the vehicle to monitoring authorities in response to a positive detection of consumption of the selected substance by the vehicle operator.

12. The method of claim 8, wherein the vehicle further includes a sobriety testing device for testing for alcohol in the breath of the vehicle operator and circuitry for disabling the operation of the vehicle in response to a positive detection of alcohol in the breath of the vehicle operator, the method further comprising:
initiating a test of the vehicle operator by the sobriety testing device when the transdermal sensor detects alcohol consumption by the vehicle operator.

13. A system for identifying a vehicle operator comprising:
a first body communications device for transmitting signals carrying information identifying a vehicle operator using the body of the vehicle operator as a communications path; and
a second body communications device for receiving signals carrying the information identifying the vehicle operator from the body of the vehicle operator.

14. The system of claim 13, further comprising a capacitive touch pad disposed on the vehicle for transmitting signals carrying the information identifying the vehicle operator from the body of the vehicle operator to the second body communications device.

15. The system of claim 13, wherein the second body communications device forms a portion of a sobriety interlock system including a sobriety testing unit, wherein the signals carrying information identifying the vehicle operator selectively enable sobriety testing of the vehicle operator by the sobriety testing unit.

16. The system of claim 13, further comprising a capacitive touch pad disposed on the sobriety testing unit for transmitting signals carrying the information identifying the vehicle operator from the body of the vehicle operator to the second body communications device.

17. The system of claim 13, wherein the first body communications unit comprises a mobile body communications unit and the second body communications unit comprises a base body communications unit.

18. The system of claim 13, wherein the system forms a portion of a sobriety interlock system including a sobriety testing unit, the sobriety testing unit comprising:
a mouthpiece for receiving a breath sample of an individual under test and including a contact for establishing an electrical coupling to the individual under test; and
an electrical measurement circuit adapted to be electrically coupled to the mouthpiece and operable to measure an electrical characteristic of a signal received from at least one of the first and second body communications devices through the body of the individual under test

19. A method of identifying an individual for selective substance testing comprising:
generating signals carrying information identifying an individual;
transmitting the signals carrying information identifying the individual using the body of the individual as a communications path;
receiving the signals carrying the information identifying the individual from the body of the individual; and
processing the received signals to determine if the individual requires testing for use of a substance.

20. The method of claim 19, further comprising requiring testing by a sobriety interlock system controlling operation of a vehicle in response to determining that the individual requires testing for use of a substance.

21. The method of claim 19, further comprising requiring testing by a home substance testing system in response to determining that the individual requires testing for a substance.

22. The method of claim 19, wherein generating signals carrying information identifying an individual comprises generating signals with a device fastened to the body of the individual.

23. The method of claim 19, wherein receiving the signals carrying the information identifying the individual from the body of the individual comprises receiving the signals through a capacitive touch pad.

24. The method of claim 20, wherein requiring testing by a sobriety testing device further comprises: coupling an electrical signal from the body of an individual taking the sobriety test to a circuit for measuring an electrical characteristic of the electrical signal from the body of the individual taking the test; measuring the electrical characteristic the electrical signal from the body of the individual taking the test; and
evaluating the measured electrical characteristic for selectively detecting circumvention of the required testing.

25. A method of monitoring for consumption of a selected substance by a vehicle operator comprising:
testing a vehicle operator for consumption of a selected substance with a first monitoring device tethered to the vehicle operator to generate first data representing first test results;
testing the vehicle operator for consumption of the selected substance with a second monitoring device associated with a vehicle being operated by the vehicle operator to generate second data representing second test results; and
transmitting the first and second data to a server for subsequent comparison.

26. The method of claim 25, wherein transmitting the first and second data comprises:
transmitting the first data with a wireless transmitter integral to the tethered device; and
transmitting the second data with a wireless transmitter associated with the vehicle.

27. The method of claim 25, wherein testing the vehicle operator with a first monitoring device comprises testing the vehicle operator with a transdermal sensor.

28. The method of claim 25, wherein testing the vehicle operator with a second monitoring device comprises testing the vehicle operator with a breath testing device forming a portion of a vehicle interlock system.

29. The method of claim 25, further comprising:
transmitting identification data identifying the vehicle operator tethered to the tethered device to a data processing system associated with the vehicle;
logging the transmitted identification data with the data processing system to generate a record recording operation of the vehicle by the vehicle operator.

30. A method of risk mitigation of a vehicle being driven by a driver under substance influence comprising:
retrieving data from at least one substance monitoring device; and
transmitting the data to a remote server for evaluation; and
running a risk assessment algorithm on the remote server; and
restricting the use of the vehicle based on the algorithm's outcome as transmitted to the vehicle's control system.

31. The method of claim 30, wherein the at least one substance monitoring device comprises a plurality of monitoring devices including a first monitoring device comprising a transdermal sensor.

32. The method of claim 30, wherein the at least one substance monitoring device comprises a plurality of monitoring devices including a first monitoring device comprising a breath analyzer.

33. The method of claim 32, wherein the plurality of monitoring devices includes a second monitoring device comprising a transdermal sensor.

34. The method claim 30, wherein vehicle movement is restricted to a geographical location as determined by a vehicle geo-location system.

35. The method of claim 30, wherein vehicle movement is restricted to a geographical location as determined by a geo-location system of a vehicle interlock system.

36. The method of claim 30 where the vehicle start is restricted to specific times of the day and days of the week.
